(19) United States
(12) Patent Application Publication
(10) Pub. No.: US 2007/0020649 A1
(43) Pub. Date: Jan. 25, 2007

(54) Chitosan capture of microorganisms for detection

(75) Inventors: Susan Tseng, Hockessin, DE (US); Siqun Wang, Wilmington, DE (US); Kristin Ruebling-Jass, Wilmington, DE (US)

Correspondence Address: PATENT GROUP;ATTN: J. KENNETH JOUNG, DLA PIPER RUDNICK GRAY CARY US LLP, 203 N. LASALLE ST., SUITE 1900, CHICAGO, IL, 60601, US

(73) Assignee: E.I. du Pont de Nemours and Company, Wilmington, DE (US)

(21) Appl. No.: 11/374,461

(22) Filed: Mar. 13, 2006

Related U.S. Application Data
(60) Provisional application No. 60/660,660, filed on Mar. 11, 2005.

(51) Int. Cl. C12Q 1/68; C12P 19/34; C12M 1/34

(52) U.S. Cl. 435/6; 435/287.2; 435/91.2

(57) ABSTRACT

Chitosan attached to a support surface is used to capture bacteria for detection purposes. Bacteria are captured with chitosan, lysed, and assayed using primers with sequences specific for nucleic acids of target species. The chitosan coated support serves as a capture device to collect or concentrate bacteria from a sample for subsequent assay.

This application claims the benefit of U.S. Provisional Application No. 60/660,660, filed Mar. 11, 2005, which is hereby incorporated by reference in its entirety.

### FIELD OF THE INVENTION

The field of invention relates generally to microbiology and the assay of microbes and in particular to capture of organisms for subsequent assay.

### BACKGROUND OF THE INVENTION

It is often desirable to assay for the presence of bacteria or other microorganisms in various clinical, food, environmental, or other experimental samples to identify contaminants or pathogens. Bacterial DNA or bacterial RNA, typically ribosomal RNA (rRNA) or in some cases messenger RNA (mRNA), may be assayed to assess the presence or absence of a bacterial species. For some important bacterial species, such specific probes can detect a particular RNA molecule belonging to that bacterial species. Such probes can detect a particular bacterial species when in the presence of other bacterial species. For example, probes may be used as primers in the RT-PCR assay method to detect a specific RNA molecule through its reverse transcription into DNA, followed by amplification of its copy DNA using the polymerase chain reaction. The entire bacterial detection process generally involves the growth of bacteria and extraction of bacterial DNA or RNA, followed by the PCR or RT-PCR reaction and product detection.

The successful detection of the presence of a particular bacterium that is present depends on the concentration of the bacterium in the sample. Generally, bacterial samples are cultured to assess viability and increase the numbers of the bacteria in the sample to assure an adequate level for detection. The culturing step requires substantial time and therefore delays acquiring the detection result.

Concentration of bacteria may allow detection using a shorter or possibly no culturing step. Methods have been developed to isolate, and thereby concentrate, specific bacterial strains by using antibodies specific to the strain. However, other concentration methods that are non-strain specific would allow a more general sampling of the microorganisms present in a sample. Once a mixed population of microorganisms is concentrated, the presence of specific strains can be determined using specific probes in genetic assays as described above.

Bacteria have been immobilized in a gel matrix for use as intact microbial enzyme activity systems. Different types of matrices were used to immobilize the Actinomycete-like organism, bacterial strain GJ 70, for performing enzyme assays in Buchinger et al. (Acta Biotechnol. 1997 17:123-130). The gel matrices used to immobilize cells included calcium alginate, carrageenan, chitosan, polyacrylamide-hydrazide, and chitosan-carboxymethylcellulose. Although the cells were immobilized in gels composed of these substances, no suggestion or demonstration was made for the capacity of any of these substances to capture bacteria from a sample for detection purposes.

Non-specific capture of microorganisms is described in Bundy and Fenselau (Anal. Chem. 2001 73:751-757) using carbohydrate and lectin protein interactions. Lectins, which are carbohydrate binding proteins, were found to be able to capture bacteria and an enveloped virus in some samples. Also lectins present on the surface of bacteria were suggested to be capture targets for five different carbohydrates: beta-D-glucose, alpha-D-mannose, and blood group antigens Lewis a, b, and x (Le^{a}, Le^{b}, Le^{x}). The Le^{a}, Le^{b}, Le^{x} groups, when attached separately to polyacrylamide backbones, were each able to capture some bacterial strains.

WO0153525 asserts that substances that serve as nutrients for microorganisms are useful as ligands to provide non-specific capture of microorganisms. The nutrient ligands described include carbohydrates, vitamins, iron-chelating compounds, and siderophores. The ligands described include monosaccharides, oligosaccharides and polysaccharides including a wide range of types such as D-mannose, D-glucose, L-fructose, N-acetyl-glucosamine, N-acetyl-galactosamine, rhamnose, galactosamine, glucuronic acid, N-acetylneuraminic acid, a-methyl-glucoside, xylose, and sorbitol. The nutrients are said to make use of receptor/ligand interactions, as well as the microorganism response to the presence of nutrients, for capture. However, even though a long list of potentially active ligands is given, WO0153525 does not list any de-acetylated carbohydrate polymers that are not nutrients for use in bacterial capture.

Still needed in the art are materials that are not based on either lectin/carbohydrate interactions or nutrient response and binding, and which capture microorganisms in a non-specific manner.

### SUMMARY OF THE INVENTION

The invention includes the use of supported chitosan for the capture of microorganisms such as bacteria, fungi, yeasts and enveloped viruses for the purpose of subsequent detection or assay. Chitosan capture does not target a specific microorganism, but serves to concentrate the target organism in a non strain-specific manner so that it may be more easily and rapidly assayed.

The present invention provides methods for detection of a microorganism strain in a sample. In one aspect, a method comprises the steps of providing a support coated with chitosan; providing a sample having at least one microorganism strain; contacting the coated support of with the sample, such that at least one microorganism strain is bound to the support; and detecting the presence of the at least one bound microorganism strain. Detection may be done by genetic or immunologic methods

In another aspect, a method comprises the steps of providing a support coated with chitosan; providing a sample containing a sample matrix and suspected of containing at least one target microorganism strain; contacting the coated support with the sample, such that the at least one microorganism strain is bound to the support; washing the sample matrix from the bound target microorganism strain; detecting the presence of said target microorganism strain by detection methods selected from the group consisting of genetic detection methods and immunologic detection methods.

In another aspect, a method comprises the steps of providing a support coated with chitosan; providing a sample containing a sample matrix and suspected of containing at least one target microorganism strain; contacting the coated support with the sample of such that at least one target microorganism strain is bound to the support; washing the sample matrix from the bound target microorganism strain; lysing the bound target microorganism strain to release nucleic acids; and detecting the presence of at least one of said nucleic acids by at least one primer directed amplification method.

In any aspect of the present invention that employs genetic detection methods, the genetic detection may be by nucleic acid hybridization or primer directed amplification. Primer directed amplification includes polymerase chain reaction, reverse transcriptase polymerase chain reaction, ligase chain reaction, strand displacement amplification or combinations of these.

In any aspect of the present methods, the support may be a bead, a film, a sheet, a particle, a filter, a membrane, a plate, a strip, a tube, a well, a gel, fibers, capillaries or any combination of any of these. The bead may be a magnetic bead, which may be about 1 to about 5 microns in diameter. Further, the support may comprise glass, silica, latex, ceramics, metals, polyolefins, polyolefin copolymers, polyolefin ionomers, polyolefin blends, poly(4-methylbutene), polystyrene, polymethacrylate, polyethylene, polypropylene, polyamide (nylon), poly(vinyl butyrate), poly(ethylenterephthalate) (PET), polyvinylchloride (PVP), polycarbonate, polyesters, orcellulose.

In any aspect of the present invention, the chitosan may be of molecular weight in the range of about 2,000 to about 500,000 Daltons and may be directly linked to the support. The chitosan may alternatively be linked to the support via a linker or attachment molecule. The attachment molecule may be one member of a binding pair independently selected from either antigen/antibody, hapten/anti-hapten, IgG/protein A, IgG/protein G, biotin/avidin, biotin/streptavidin; glutathione-S-transferase/glutathione or folic acid/folate binding protein. In any aspect of the invention, the linker may p-toluenesulfonate and the attachment molecule streptavidin.

In any aspect of the present invention, the sample may contain a plurality of microorganism strains. When such a plurality is present in the sample, any one strain may be detected independently of any other strain. The microorganism strain may be of bacteria, fungi, yeasts or enveloped viruses. The strain may be a pathogen, which may be selected from the group of genera consisting of Listeria, Escherichia, Salmonella, Shigella, Campylobacter, Clostridium, Helicobacter, Mycobacterium, Staphylococcus, Camplobacter, Enterococcus, Bacillus, Neisseria, Shigella, Streptococcus, Vibrio, Yersinia, Bordetella, Borrelia, and Pseudomonas.

In any aspect of the present invention, the sample may be a medical, environmental, food, feed, clinical or laboratory sample. Environmental samples may come from different environments, including soil, water, a medical environment, a veterinary environment, a food environment, a food preparation environment, an industrial environment, a terrorism suspect environment, and a laboratory environment.

The present invention also includes a microorganism capture device that is used in any method of the present invention and which comprises a support that includes chitosan. The present invention also includes a diagnostic kit, which includes any aspect of a capture device of the present invention.

### BRIEF DESCRIPTION OF THE FIGURES

FIG. 1 shows dissociation curves of the PCR amplified products of Listeria genus assays carried out on Listeria innocua captured with chitosan-biotin-streptavidin beads, as well as streptavidin and anti-Listeria IgG control beads.

FIG. 2 shows dissociation curves of the PCR amplified products of E. coli ATCC 25922 carried out on cells captured with chitosan-biotin-streptavidin beads.

FIG. 3 shows dissociation curves of the PCR amplified products of Listeria genus assays carried out on mixed, as well as individual, cultures of Listeria innocua and Bacillus subtilis captured with chitosan-biotin-streptavidin beads.

FIG. 4 shows dissociation curves of the PCR amplified products of Listeria genus assays carried out on Listeria innocua captured with chitosan-biotin-streptavidin beads having different sizes of chitosan attached.

FIG. 5 shows a comparison of Ct values for PCR reactions for Listeria innocua captured with directly and indirectly conjugated chitosan—Dynal magnetic beads, at two different cell concentrations.

FIG. 6 shows dissociation curves of the PCR amplified products of Listeria genus and Listeria monocytogenes assays carried out on Listeria monocytogenes captured with chitosan-biotin-streptavidin beads.

### DETAILED DESCRIPTION OF THE INVENTION

The present invention includes a method for detecting a microorganism in a sample that makes use of a support coated with chitosan to capture microorganisms in the sample. Also the present invention includes a device for capturing microorganisms that includes a support coated with chitosan. Capture using chitosan is not specific to any strain, species, or type of microorganism and therefore it provides for the concentration of a general sampling of microorganisms in a sample. Specific strains of microorganisms can then be detected from among the captured population using any known analysis with strain-specific probes. Detection of microbial contaminants or pathogens in clinical, food, environmental, or other experimental samples is possible using this method. Of particular interest is the detection of food borne pathogens, particularly bacteria, although the person of skill in the art will recognize that the invention is not so limited.

The following are to be used in interpreting the scope and meaning of the claims and the specification.

Abbreviations

| | |
|---|---|
| Polymerase chain reaction | PCR |
| reverse transcription - polymerase chain reaction | RT-PCR |
| Ribonucleic acid | RNA |
| Deoxyribonucleic acid | DNA |
| p-toluenesulfonate | tosyl |

Definitions

As used herein, “RNA” refers to a nucleic acid molecule comprising a ribose sugar as opposed to a deoxyribose sugar as found in DNA. RNA will refer to all species of RNA including messenger RNA (mRNA), ribosomal RNA (rRNA), transfer RNA (tRNA) as well as small RNA species that have regulatory function. “Small RNA species” have a specific meaning and refer to untranslated RNAs with housekeeping or regulatory roles in bacteria. “Small RNA species” are not rRNA or tRNA.

As used herein, “reverse transcription followed by polymerase chain reaction”, or “RT-PCR”, refers to a technique for synthesizing and amplifying a DNA molecule having a sequence that is a copy of an RNA sequence. RT-PCR is useful for detecting RNA species such as in microbe detection, quantitative analysis of gene expression, as well as for producing DNA copies of RNA for use in cloning, cDNA library construction, probe synthesis, and signal amplification in in situ hybridizations. The technique consists of two parts: synthesis of cDNA from RNA by reverse transcription (RT), and amplification of a specific cDNA by polymerase chain reaction (PCR). Reverse transcriptase is an RNA dependent DNA polymerase that catalyses the polymerization of nucleotides using template RNA or the RNA molecule in an RNA:DNA hybrid.

As used herein, “primer” refers to an oligonucleotide (synthetic or occurring naturally), which is capable of acting as a point of initiation of nucleic acid synthesis or replication along a template strand when placed under conditions in which synthesis of a complementary stand is catalyzed by a polymerase. Within the context of reverse transcription, primers are composed of nucleic acids and prime on RNA templates. Within the context of PCR, primers are composed of nucleic acids and prime on DNA templates.

As used herein, “amplification product” refers to nucleic acid fragments that are produced during a primer directed amplification reaction. Typical methods of primer directed amplification include polymerase chain reaction (PCR), RT-PCR, ligase chain reaction (LCR) or strand displacement amplification (SDA).

As used herein, “thermocycling” refers to the entire pattern of changing temperature used during an RT-PCR or PCR assay. This process is common and well known in the art (see for example Sambrook Supra; and U.S. Pat. No. 4,683,202 (1987, Mullis, et al.) and U.S. Pat. No. 4,683,195 (1986, Mullis, et al.).

In general, PCR thermocycling includes an initial denaturing step at high temperature, followed by a repetitive series of temperature cycles designed to allow template denaturation, primer annealing, and extension of the annealed primers by the polymerase. Generally, the samples are heated initially, for example, for 2-10 minutes to denature the double stranded DNA. Then, in the beginning of each cycle, the samples are denatured, typically for 20 to 60 seconds, depending on the samples and the type of instrument used. After-denaturing, the primers are allowed to anneal to the target DNA at a lower temperature (preferably from about 40° C. to 72° C. for about 20 to 60 sec). Extension of the primer(s) by the polymerase is often carried out at a temperature ranging from about 65° C. to 72° C. The amount of time used for extension will depend on the size of the amplicon; preferably, it is about 1 minute for 1 kb of DNA to be amplified. In addition, the annealing can be combined with the extension step, resulting in a two step cycling. The RT-PCR thermocycling pattern includes an initial incubation in the range of about 30° C. to 70° C. for about 10 to 15 min for the reverse transcription reaction.

Thermocycling may include additional temperature shifts used in RT-PCR and PCR assays.

As used herein, “chitosan” refers to the polysaccharide poly-[1-4]-β-D-glucosamine that comes in a variety of forms including, but not limited to, mixtures of different molecular weight molecules, for example, with species that preferably range between 2,000 and 500,000 Daltons, and preparations with different degrees of deacetylation, generally ranging between about 30 and 99.9%.

As used herein, “support” refers to any material that can be at least partially covered with chitosan, and thus is useful in the chitosan mediated capture of microorganisms. A support with a chitosan covering forms the basis of a microorganism capture device. Supports may take a variety of shapes and may be composed of a variety of type of materials.

As used herein, “coated” refers to having a coating material on a support. The coating material may totally, or partially cover the support.

As used herein, “sample matrix” refers to components of a sample other than microorganisms.

As used herein, “detecting” refers to the identification of a component of a microorganism which thereby determines that the microorganism is present.

As used herein, “genetic detection” refers to the identification of a component of genetic material such as DNA or RNA that is derived from target microorganisms. Genetic detection assays include, for example, PCR and RT-PCR.

As used herein, “immunological detection” refers to the identification of an antigenic material such as a protein or a proteoglycan that is derived from target microorganisms. Typical molecules used in such detection can be antibodies, polypeptides selected from processes such as phage display, or aptamers from a screening process.

As used herein, “attachment molecule” refers to a molecule that can act as an intermediary molecule in the sequestering of chitosan to a surface. The attachment molecule is attached to a surface and interacts with chitosan, or a chitosan derivative to maintain chitosan at the surface.

As used herein, “primer directed amplification” refers to a method of genetic detection that makes use of a nucleic acid primer to act as a point of initiation of nucleic acid synthesis. The synthesis proceeds using a nucleic acid template such that the sequence complementary to the template is produced. This reaction increases the number of copies of the sequence, which is termed amplification. This increase fosters sequence detection. Two primers may be used that are complementary to hybridizing DNA strands such that the sequence between the two primers is amplified.

As used herein, “microorganism” means any cell having genetic material suitable for analysis or detection. An enveloped virus may also be considered a microorganism. “Target microorganism” refers to any microorganism that is desired to be detected. An assay specifically designed to detect the target microorganism may be used to determine its presence.

As used herein, “binding partner” refers to one of two molecules that are able to interact with each other such that they form a complex. A “binding pair” consists of two binding partners. The term “binding pair” includes any of the class of immune-type binding pairs, such as antigen/antibody or hapten/anti-hapten systems; and also any of the class of nonimmune-type binding pairs, such as biotin/avidin; biotin/streptavidin; folic acid/folate binding protein; glutathione-S-transferase/glutathione; 6× histidine Tag/Ni-NTA; complementary nucleic acid segments; and protein A or G/immunoglobulins.

As used herein, “bound to the support” refers to the state of microorganisms which are captured by chitosan that is itself coated on a support.

As used herein, “microorganism strains” are multiple types of microorganisms which are distinguishable through a detection method. Different microorganism strains may be, for example, of different genera, different species within a genera, and different isolates within a species.

As used herein, “lysing” refers to the breaking open of cells such that the cell contents is accessible. Lysis methods may include, for example, treatments such as sonication, osmotic shock, high temperature (from about 50° C. to about 100° C.), and incubation with an enzyme such as lysozyme, glucolase, zymolose, lyticase, proteinase K, proteinase E and viral enolysins.

Chitosan

The method of the present invention uses chitosan, coated on a support, to capture microorganisms for detection. A chitosan-coated support provides a device for microorganism capture. Chitosan is the commonly used name for poly-[1-4]-β-D-glucosamine. Chitosan is chemically derived from chitin which is a poly-[1-4]-β-N-acetyl-D-glucosamine which, in turn, is derived from the cell walls of fungi, the shells of insects and, especially, crustaceans. Chitin is treated with strong alkalis to remove acetyl groups, which results in chitosan, a commercially available product. Depending on the specific treatment of chitin, the resulting chitosan may vary in its degree of deacetylation. Commercially available chitosan preparations typically have at least 90% deacetylation. These commercial preparations, as well as preparations with deacetylation of 30% to 99.9%, preferably about 50% or greater, may be utilized in the present invention. Deacetylation leaves free amine groups which are positively charged.

Chitosan is generally insoluble in water, but dissolves in dilute solutions of organic acids such as acetic, formic, tartaric, and citric acids and also in dilute mineral acids such as sulfuric acid. Inorganic acid solutions of chitosan are also possible. Preparations of unusually short chitosan polymers, of low molecular weight that is less than about 10,000 Daltons, are soluble in water. Chitosan molecules with molecular weights of about 2,000 to about 500,000 Daltons may be used in the present invention. Typically, chitosan preparations with varying molecular weights of individual species ranging between about 8,000 and about 500,000 Daltons are available and are useful in the present invention. Preferred are preparations with sizes of about 34,000 Daltons to about 180,000 Daltons.

Support

A support in the present invention is any material which provides a surface to which chitosan can be attached, and can then be used for capturing microorganisms. The support may be in the form of, for example, a film, a sheet, a gel, a filter, a membrane, fibers, a strip, a dipstick, a plate that is flat or has wells, a tube, a particle, capillaries and a bead, which includes styrene and magnetic types. Usable magnetic beads for example, may be comprised of a magnetic elemental ion in a polymer matrix, or they may include chromium dioxide or other magnetic compounds. Preferred magnetic beads are superparamagnetic beads such as polystyrene spheres having an even dispersion of a mix of maghemite (gamma-Fe₂O₃) and magnetite (Fe₃0₄), and a thin polystyrene shell. Supports may be of different sizes. Supports that have at least one dimension measured to be between about one half and 500 microns are particularly useful for small-scale capture and assay. Preferred are beads of sizes in the range of about 1 micron to about 5 microns. In addition, nanoparticles may be used in supports in the present invention.

The material of the support may be in any state that allows attachment of chitosan and forms a substrate for the chitosan such as solid, porous, a gel, a matrix, and a nonwoven. Materials that may comprise the support include, for example, glass, silica, latex, ceramics, and metals. In addition the support may be made of one or more polymers including, but not limited to, polyolefins such as polyethylenes and polypropylenes, polyolefin copolymers, ionomers and blends, poly(4-methylbutene), polystyrene, polymethacrylate, polyethylene, polypropylene, polyamide (nylon), poly(vinyl butyrate), poly(ethyleneterephthalate) ( PET), polyvinylchloride (PVP), polycarbonate, polyesters, and cellulose.

Chitosan Coating and Capture Device

A support surface coated with chitosan has chitosan attached to the surface such that the support is partially or fully covered. Chitosan may be attached to a support surface directly or indirectly. Various methods are known for coating chitosan directly onto the surfaces of polymer materials that result in covalent, ionic, or other types of attachment. Some examples of chitosan attachment to polymers are crosslinking as in PCT application WO 00/49219, graft polymerization as in Japanese Kokai 05269181, using acid surface treatments as in U.S. Patent Application 2003/0091612 and U.S. patent application Ser. No. 10/288762, using base and acid surface treatments as in co-pending U.S. patent application 10/205660, and reacting with amino-reactive functional groups as in co-pending U.S. Patent Application 60/496296. Examples of amino-reactive groups include positively charged species such as metal ions, anhydrides, carboxylic acids, isocyanates, epoxides, acid chlorides, and enones.

Chitosan may be attached to a surface through a linking agent. For example, p-toluenesulfonate (abbreviated tosyl) is a highly reactive leaving group that may be attached to surfaces and is used to couple primary amines. Tosyl-activated surfaces, such as agarose or magnetic beads, may be used in the attachment of chitosan. Amine reactive groups such as isotriocyanates, succinimidyl esters and sulfonyl halides, as well as sulfhydryl reactive groups including maleimides and haloacetyl derivatives, may also be used to link chitosan to surfaces.

Chitosan may also be attached to a support surface through interaction with an attachment molecule. The attachment molecule may be a member of a binding pair that is used to secure chitosan to a surface. For example, chitosan may be derivatized with biotin which then binds to streptavidin, the second member of this binding pair, that has been previously attached to a surface. Thus, a chitosan-biotin-streptavidin surface attachment is made. Other binding pairs where one member is amenable to attachment to chitosan, and the second member may be attached to a surface, may be used in preparing the coated surface. Some additional examples of binding pairs are any of the class of immune-type binding pairs, such as antigen/antibody or hapten/anti-hapten systems; and also any of the class of nonimmune-type binding pairs, such as biotin-avidin, folic acid-folate binding protein; glutathione-S-transferase-glutathione; 6× histidine Tag-Ni-NTA; complementary nucleic acid segments; and protein A- or G-immunoglobulins.

One skilled in the art will readily appreciate how to employ the above-described, as well as other well-known, methods of attachment to coat the surface of a support with chitosan. A preferred support surface for coating with chitosan, for use in the invention, is that of micrometer scale magnetic beads, such as for example beads of about 1 to about 5 micrometers. Preferred methods for attaching chitosan to the beads are using tosyl linking groups and using the streptavidin-biotin binding pair. Using either of these coating methods, beads were prepared and used to capture bacteria that were then successfully assayed, as described in the Examples.

The amount of chitosan coated support that is required to capture microorganisms for successful detection is readily determined by one skilled in the art. Generally, different amounts of chitosan coated support are used for capture, followed by detection assay testing. For example, the chitosan coated beads were used at successive dilutions for bacterial capture. Detection assays of the bacteria in different capture bead dilution samples, in Example 6, showed that all dilutions provided detection signals, and the undiluted bead capture samples provided the strongest detection signals. The chitosan coated support used for capture of microorganisms for detection may be provided as a capture device.

The device may take any form which is amenable to sample contact and microorganism capture. The device may be in the form of, for example, at least one dipstick, film, filter, tube, well, or plate, or it may be composed of particles or beads. In one embodiment, chitosan can be used to coat the channels of a microfluidic device. Various configurations can be applied as well, for example, membranes coated with chitosan, beads coated with chitosan and other solid surfaces. Upon passing through, microorganisms can bind and separate from food matrixes and other environmental contaminants providing a pure sample for down stream analysis.

Chitosan Capture of Microorganisms

Chitosan is a positively charged molecule, which makes it amenable to interaction with the surfaces of many microorganisms, since their surfaces are generally negatively charged. The positive/negative charge surface interactions are thought to be involved in the antimicrobial activity of chitosan, leading to death of microbes by an as yet unspecified mechanism. Binding of polycationic molecules has been shown to disrupt the integrity of the outer membrane resulting in loss of the barrier function. (I. M. Helander et., al, 2001, International Journal of Food Microbiology 71(2-3): 235-244). This antimicrobial property of chitosan directs one away from using chitosan as a microorganism capture agent for use in detection, since death of the microorganism will lead to cell disintegration and loss of the cell material required for detection. However, the present method takes advantage of the positive/negative charge surface interactions to capture microorganisms and surprisingly, the cell contents required for detection are maintained.

The capture of microorganisms using chitosan is not specific to a particular strain or type of microorganism, that is, it is non-strain specific. Using the present method results in the capture by chitosan of a population of different microorganisms present in a sample. The population of microorganisms captured with chitosan is generally representative of the population of microorganisms in the sample, except that a microorganism with no negative surface charge would not be included. However, the majority of microorganisms in the sample would be represented in the chitosan captured population.

Ultimately, this property of chitosan for non-strain specific capture of microorganisms results in a general capture system that allows for multiple microorganisms to be targeted for assay in the same captured sample. For example, in assaying for contamination of food samples, it may be desired to test for Listeria monocytogenes, E. coli O157:H7 and Salmonella all in the same sample. A single chitosan capture step may then be followed by PCR or RT-PCR assays using specific primers to amplify different nucleic acid sequences from each of these microorganisms. Thus separate sample handling and preparation for each strain may not be necessary.

Capture of microorganisms using a chitosan coated support may be carried out by various methods known to one skilled in the art where contact occurs between the microorganisms in a sample and the chitosan on the support. To capture microorganisms in a sample, for example, a dipstick coated with chitosan may be immersed in a sample solution, a sample solution may be poured onto a film coated with chitosan, a sample solution may be poured into a tube or well coated with chitosan, or a sample solution may be passed through a filter coated with chitosan. In any capture method it is optional to include mixing and/or incubation steps to increase the microorganism contact with the chitosan that is coated on the support. A preferred capture method is mixing and incubating a microorganism-containing sample with chitosan coated beads. When microorganisms are captured by chitosan which is coated on a support, the microorganisms are bound to the support through the chitosan.

Microorganisms

Microorganisms with negatively charged surfaces may be detected by the present invention including, for example, bacteria, fungi, yeasts and enveloped viruses. The present method has utility in the detection of pathogens, which may be critical for food safety or for medical, environmental or anti-terrorist reasons. It is particularly useful in the detection of pathogenic bacteria, including both gram negative and gram positive bacteria, various yeasts, molds, and mycoplasmas. Genera of target microbes to be detected include, but are not limited to, Listeria, Eschedchia, Salmonella, Campylobacter, Clostridium, Helicobacter, Mycobacterium, Staphylococcus, Shigella, Enterococcus, Bacillus, Neisseria, Shigella, Streptococcus, Vibrio, Yersinia, Bordetella, Borrelia, and Pseudomonas. Some specific microorganism strains that are targets for detection include Escherichia coli O157: H7, Yersinia enterocolitica, Yersinia pseudotuberculosis, Vibrio cholerae, Vibrio parahaemolyticus, Vibrio vulnificus, Listeria monocytogenes, Staphylococcus aureus, Staphylococcal enterotoxin ssp, Bacillus cereus, Bacillus anthracis, Clostridium perfringens, and Clostridium botulinum.

Samples

The present invention includes an improved method for the detection of microorganisms that may be applied to a variety of samples. Different types of samples that may harbor microorganisms that require detection include, but are not limited to, medical, environmental, food, feed, and laboratory samples. Medical samples may include cells, tissues or fluids from a biological source such as a human or an animal that are assayed for clinical diagnosis. Environmental samples may be, for example, from a medical facility, an industrial facility, soil, a water source, a food preparation area, or a potentially contaminated area such as by bioterrorism. Food processing, handling and preparation area samples are preferred, as these are of particular concern for the spread and contamination of bacterial pathogens in the food supply.

Samples obtained in the form of a liquid may be used directly, or may be concentrated, for example, by centrifugation. Samples in the form of a solid or a semi-solid may be extracted by a method such as rinsing. Samples may be taken from surfaces such as by swabbing or rinsing. In all of these cases, chitosan capture may be used in addition to or in replacement of other methods of concentration. The chitosan capture of microorganisms isolates them from the sample in a concentrated state. The capture also allows the isolation of microorganisms from sample matrix components that may inhibit the detection procedures that are used following capture. Optionally, cultures may be grown from collected samples prior to chitosan capture.

Detection of microorganisms

The detection process following microorganism capture optionally includes washing to remove sample matrix components.

Microorganisms that have been captured may be detected by immunologic or genetic detection methods. Immunological detection is detection of an antigenic material derived from the target organism, which is commonly a biological molecule acting as a marker on the surface of bacteria or viral particles. The detection is directed to a protein, proteoglycan, or other material with antigenic activity. Detection of the antigenic material is typically by an antibody, a polypeptide selected from a process such as phage display, or an aptamer from a screening process. Immunological detection methods are well known to those skilled in the art, such as immunoprecipitation, and enzyme-linked immunosorbent assay (ELISA). Antibody binding can be detected in several ways including by labeling either the primary or the secondary antibody with a fluorescent dye, labeling the primary or the secondary antibody with a quantum dot, labeling the primary or the secondary antibody with an enzyme that can produce chemiluminescence or a colored substrate. Devices used for detecting the binding event may be either a plate reader or a lateral flow device.

Preferred is detection by genetic assay. The captured microorganisms are lysed to render their genetic material available for assay. Lysis methods are well known to one skilled in the art, and include treatments such as sonication, osmotic shock, high temperature (from about 50° C. to about 100° C.), and incubation with an enzyme such as lysozyme, glucolase, zymolose, lyticase, proteinase K, proteinase E and viral enolysins.

Most commonly used genetic detection assays detect the nucleic acids of a specific microorganism, including the DNA and/or RNA. The stringency of conditions used in a genetic detection method correlates with the level of variation in nucleic acid sequence that is detected, as is known to one skilled in the art. Highly stringent conditions of salt concentration and temperature limit the detection to the exact nucleic acid sequence of the target. Thus microorganism strains with small variations in a target nucleic acid sequence may be distinguished using a highly stringent genetic assay. Genetic detection may be based on nucleic acid hybridization where a single stranded nucleic acid probe is hybridized to the denatured nucleic acids of the microorganism such that a double stranded nucleic acid is produced, including the probe strand. One skilled in the art will be familiar with probe labels, such as radioactive, fluorescent, and chemiluminescent labels, for detecting the hybrid following gel electrophoresis, capillary electrophoresis, or other separation method.

Particularly useful genetic detection methods are based on primer directed nucleic acid amplification. Primer directed nucleic acid amplification methods include, for example, thermal cycling methods (e.g., polymerase chain reaction (PCR) and ligase chain reaction (LCR)), as well as isothermal methods and strand displacement amplification (SDA). The preferred methods are PCR and RT-PCR (reverse transcriptase-PCR). Typically, in PCR-type amplification techniques, the primers have different sequences and are not complementary to each other. Depending on the desired target microorganism for detection, the sequences of the primers are designed to provide for both efficient and faithful replication of the target nucleic acid which is specific for the single strain of microorganism or class of microorganisms to be detected. Methods of PCR primer design are common and well known in the art (Thein and Wallace, “The use of oligonucleotide as specific hybridization probes in the Diagnosis of Genetic Disorders”, In Human Genetic Diseases: A Practical Approach, K. E. Davis Ed., (1986) pp 33-50; IRL: Herndon, VA; and Rychlik, W. (1993) In White, B. A. (ed.), Methods in Molecular Biology, Vol. 15, pp 31-39, PCR Protocols: Current Methods and Applications. Humania: Totowa, N.J.).

In RT-PCR, an initial reaction with a reverse transcriptase, such as HIV Reverse Transcriptase (Ambion), Transcriptor Reverse Transcriptase (Roche), or Thermoscript Reverse Transcriptase (Invitrogen) creates a DNA copy from an RNA template using a hybridizing primer. More than one primer may be included if it is desired to make DNA copies from more than one target RNA. RT and PCR may be carried out in one step, or in sequential steps. A sample of this reaction may be transferred to another assay tube containing a pair of primers that initiate synthesis of the desired segment of DNA from the reverse transcribed template. Thermostable DNA polymerases for use in this method include, for example, Taq polymerase, Pfu, Vent, and Sequitherm DNA Polymerase (EPICENTRE). More than one pair of primers may be included if synthesis of multiple segments of DNA is desired. Also a single new primer may be added that will amplify a DNA segment with the original RT-PCR primer as the second primer of the pair. One skilled in the art will know conditions of temperature and incubation times required in thermocycling these reactions to produce the amplified nucleic acid product.

The method of detection of the amplified product is not limited in the instant invention. Any method whereby the product of the amplification reaction is detected may be used. Some methods for product detection include gel electrophoresis separation and ethidium bromide staining, or detection of an incorporated fluorescent label or radiolabel in the product. Methods that do not require a separation step prior to detection of the amplified product may also be used. These methods are commonly referred to as real time PCR or homogeneous detection. Most real time methods detect amplified product formation by monitoring changes in fluorescence during thermocycling. These methods include but are not limited to: TaqMan® dual labeled probes (Applied Biosystems, Foster City, Calif. 94404), Molecular Beacons (Tyagi S and Kramer FR, 1996, Nat Biotechnol 14, 303-308), and SYBR® Green dye (Molecular Probes, Inc Eugene, Oreg. 97402-0469). Some of these same homogeneous methods can be used for end point detection of amplified products as well. An example of this type of method is SYBR® Green dye dissociation curve analysis. In dissociation curve analysis a final slow ramp in temperature, generally about 60° C. to 90° C., combined with fluorescence monitoring can detect the melting point and thereby the presence of an amplified product (Ririe et al., 1997, Anal. Biochem. 245:154-60).

Diagnostic Kit

Typically, a diagnostic kit of the invention includes a capture device having chitosan coated on a support as described above, a lysis agent, and genetic detection assay components. A preferred lysis agent is a lytic enzyme supplied in a buffer, and preferred genetic detection assay components include one or more primers specific for a target microorganism.

### EXAMPLES

The present invention is further illustrated in the following Examples. It should be understood that these Examples, while indicating preferred embodiments of the invention, are given by way of illustration only.

The meaning of abbreviations used is as follows: “sec” mean second(s), “min” means minute(s), “hr” means hour(s), “μl” means microliter(s), “ml” means milliliter(s), “nm” means nanometer(s), “mm” means millimeter(s), “cm” means centimeter(s), “μM” means micrometer(s), “mM” means millimolar, “M” means molar, “mmol” means millimole(s), “μmole” means micromole(s), “g” means gram(s), “μg” means microgram(s), “mg” means milligram(s), “g” means the gravitation constant, “rpm” means revolutions per minute, “Ct value” means Cycle threshold value, and “cfu” means colony forming units.

Standard recombinant DNA and molecular cloning techniques used in the Examples are well known in the art and are described by Sambrook, J., Fritsch, E. F. and Maniatis, T., Molecular Cloning: A Laboratory Manual, Cold Spring Harbor Laboratory Press, Cold Spring Harbor, N.Y., 1989, by T. J. Silhavy, M. L. Bennan, and L. W. Enquist, Experiments with Gene Fusions, Cold Spring Harbor Laboratory, Cold Spring Harbor, N.Y., 1984, and by Ausubel, F. M. et al., Current Protocols in Molecular Biology, Greene Publishing Assoc. and Wiley-Interscience, N.Y., 1987. Materials and methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, D.C., 1994, or by Thomas D. Brock in Biotechnology: A Textbook of Industrial Microbiology, Second Edition, Sinauer Associates, Inc., Sunderland, Mass., 1989.

All reagents and materials used for the growth and maintenance of bacterial cells were obtained from Aldrich Chemicals (Milwaukee, Wis.), or Sigma Chemical Company (St. Louis, Mo.), unless otherwise specified.

### Example 1

### Indirect Attachment of Chitosan to Magnetic Beads with Biotin/Streptavidin and use for Capture of Listeria innocua for Detection Assay

Chitosan was derivatized with biotin to mediate attachment to streptavidin magnetic beads as follows. Chitosan (M) was purchased from Primex (Siglufjordur, Iceland). The actual average size of the chitosan in this preparation was determined to be 75,000 Daltons using HPLC gel permeation chromatography-multi angle laser light scattering (GPC-MALLS; Anthonsen et al., (1994) Carbohydrate Polymer 25: 13-23). Chitosan was prepared as a 1% solution in 0.5% acetic acid. A solution of 1 Omg/ml Biotin-LC-NHS (Pierce Biotechnology Inc., Rockford, Ill.) was freshly made in DMSO and 30 μl was added to 0.5 ml of the chitosan solution. This mixture was incubated at room temperature for 1 hr with rocking. The biotin labeled chitosan was purified using a Qiagen spin column (Valencia, Calif.). The concentration of the collected excluded fraction of chitosan-biotin solution was determined by measuring absorption at 258 nm to be approximately 5 mg/0.6 ml.

Streptavidin/Dynal 2.8 μM magnetic beads were purchased from Dynal Biotech ASA (Oslo, Norway). Since the recommendation of the manufacturer is that about 5-10 μg of IgG-biotin can be conjugated to 1 mg of streptavidin/Dynal 2.8 μM beads, it was estimated that˜100 μg of chitosan-biotin would saturate all streptavidin sites on 10 mg of beads. 10 mg of streptavidin/Dynal beads were washed three times with PBS buffer pH 7.4, with magnetic recovery of the beads between each wash. After washing, the beads were resuspended in 1 ml of PBS buffer pH 7.4 and 12 μl (˜100 μg) of purified chitosan-biotin was added. This mix was incubated at room temperature for 1 hr with rocking. The chitosan-biotin-streptavidin beads were separated using a magnetic rack, then washed and resuspended in PBS, pH 5.75. 1 mg/ml of BSA was added for storage. Listeria innocua ATCC #BM680 culture stock was purchased from the ATCC. Listeria innocua, a gram positive bacterium, was inoculated into BHI broth (Brain Heart Infusion; Teknova, Hollister, Calif.) and grown overnight at 37° C. 1:10 serial dilutions were made in BHI broth and 100 μl of each was plated in duplicate on BHI plates, grown overnight at 37° C., and colonies counted to determine cfu/ml.

Three samples each of 1 ml of Listeria innocua culture at 1.3×10² cfu/ml were transferred to 2 ml screw cap vials. To one vial was added 30 μl of the chitosan-biotin-streptavidin magnetic beads prepared above. As controls, to the second vial 30 μl of anti-Listeria IgG Dynal magnetic beads (Dynal Biotech ASA, Oslo, Norway) was added and to the third vial 30 μl of streptavidin/Dynal beads was added. The samples were mixed by inverting and incubated at room temperature with rocking for 10 min. The beads were separated using a magnetic rack, washed once with 1 ml of PBS buffer pH 7.4, and resuspended in 120 μl of Qualicon™ lysis/ProE buffer (Qualicon, Wilmington, Del.). The samples were incubated at 55° C. in a heating block for 15 min, then at 95° C. for 10 min to complete lysis and inactivate the ProE.

The presence of Listeria innocua was assayed using a Listeria genus BAX™ PCR detection kit from Qualicon (Wilmington, Del.) according to the manufacturer's instructions. Magnetic beads in the cell lysate samples were separated magnetically and discarded. 50 μl of lysate was transferred to a PCR tube containing a Listeria PCR Tablet. The tubes were sealed, vortexed until the tablets dissolved, and assayed in an ABI Prism® 7500 Sequence Detector (Applied Biosystems, Foster City, Calif.) using the “Listeria genus ” program with a 50 μl sample size. Conditions were Stage l: 95° C. for 10 min, 1 cycle; Stage II: 95° C. for 15 sec, 68° C. for 3 min, 84° C. for 35 sec, 39 cycles. The Melting Curve program was run following the PCR amplification. Conditions were Stage l: 70° C. for 2 min, 1 cycle; Stage II: start at 60° C. and continuously increase 0.3° C. per step, each step recording for 35 sec, 98 cycles.

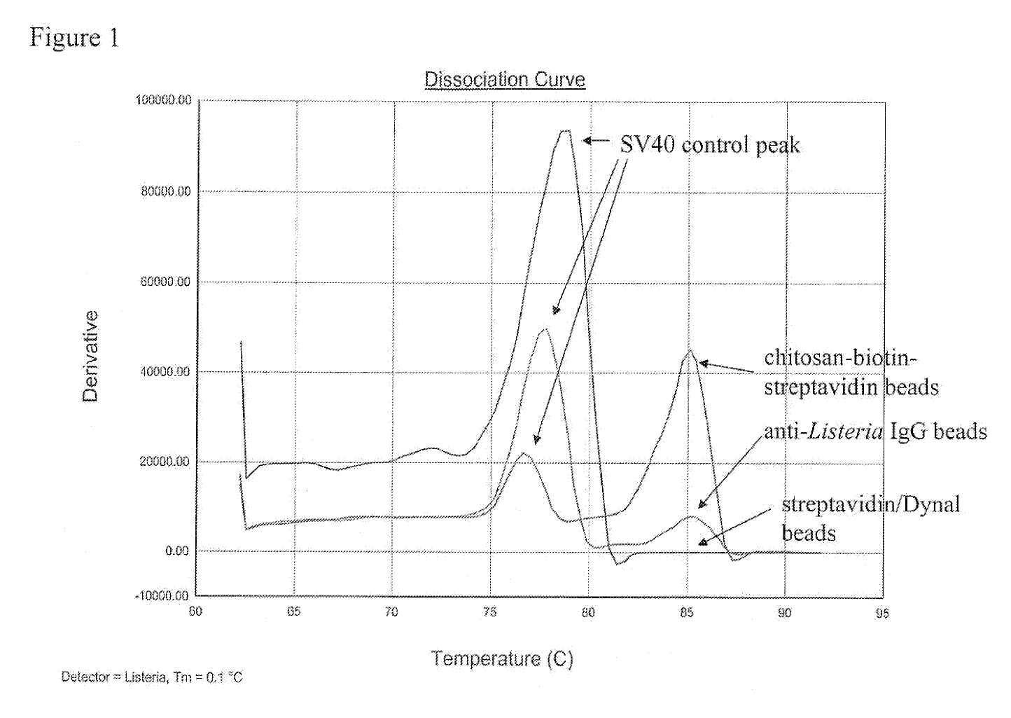

FIG. 1 shows the results of the Listeria detection assay following chitosan capture and PCR amplification as dissociation (melt) curves. Each of the three samples showed a peak at about 77.5° C. which comes from control SV40 DNA in the PCR kit. Results showed an additional peak ranging from 83.7° C. to 85.6° C. in the chitosan-biotin-streptavidin bead capture sample and the anti-Listeria IgG bead capture sample which represented amplified Listeria DNA. This peak was not present in the control streptavidin bead capture sample indicating that no Listeria cells were captured by these beads. This peak is much larger in the chitosan-biotin-streptavidin bead capture sample than in the anti-Listeria IgG bead capture sample, which indicated that chitosan capture of Listeria was even more effective than capture using anti-Listeria IgG.

### Example 2

### Capture of E. coli using Chitosan-biotin-streptavidin Magnetic Beads and Detection

E. coli strain ATCC 25922 was inoculated into BHI broth and grown overnight at 37° C. 1:10 serial dilutions were made in BHI broth and 100 μl of each was plated in duplicate on BHI plates, grown overnight at 37° C., and colonies counted to determine cfu/ml. Two samples of the E. coli culture at 10⁴ cfu/ml were treated with chitosan-biotin-streptavidin magnetic beads, prepared as in Example 1, and the samples were captured as described in Example 1. The samples were then washed as described in Example 1 and the cells were lysed at 37° C. for 10 min in Qualicon™ lysis/ProE buffer. The presence of E. coli was assayed using the commercially available E. coli PCR reagent which includes 16S-518 3F and 16S-579 3R primers (Applied Biosystems, Foster City, Calif.). 2 μl of the lysate was added to 48 μl of E. coli PCR reagent. Amplification reactions were performed and assayed in an ABI Prism® 7500 Sequence Detector as described in Example 1 except that Stage II used 95° C. for 15 sec, 60° C. for 1 min, 78° C. for 35 sec, 40 cycles.

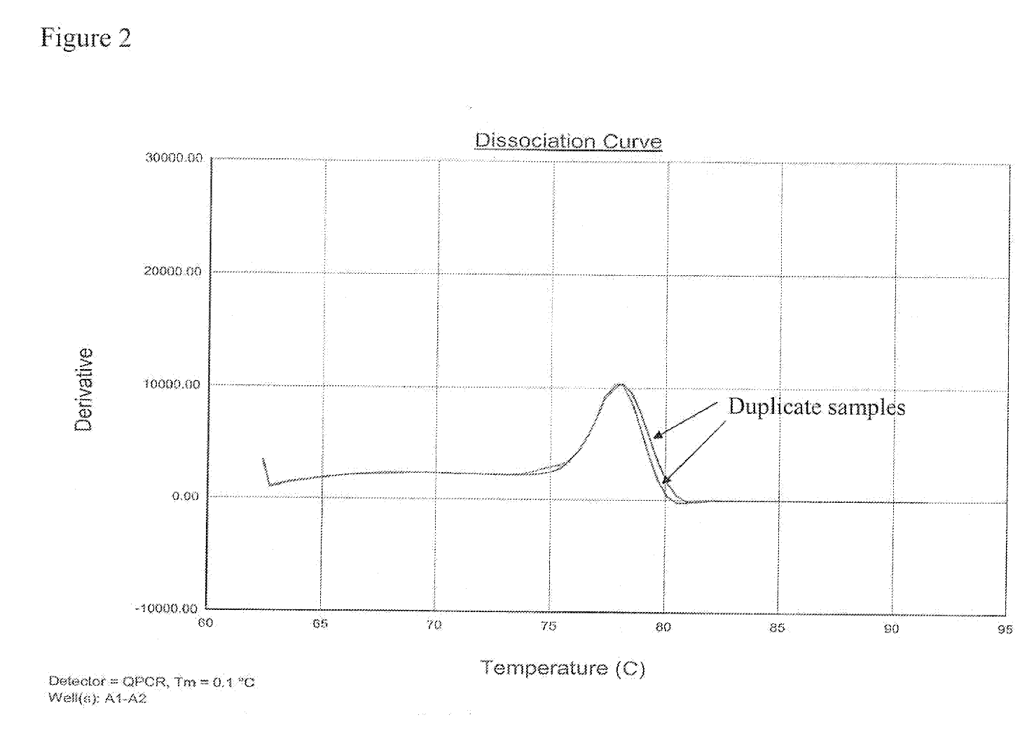

FIG. 2 shows the results of the E. coli detection assay following chitosan capture as dissociation (melt) curves. Results showed a peak ranging from 76.7° C. to 78.6° C. which represented amplified E. coli DNA. This result demonstrates the capture of E. coli, a gram negative bacterium, using the chitosan-biotin-streptavidin beads.

### Example 3

### Capture of Listeria innocua from a Mixed Culture using Chitosan-biotin-streptavidin Magnetic Beads, and Detection

Listeria innocua (ATCC #BM680) was inoculated into BHI broth and grown overnight at 37° C. Bacillus subtilis (ATCC #6633) was similarly grown.

Each culture was diluted and assayed as described in Example 1 to determine cfu/ml. 100 μl of the diluted Listeria culture at 10³ cfu/ml was added to 0.9 ml of the Bacillus culture at 10⁸ cfu/ml. 100 μl of the two separate cultures ( at˜10² cfu/ml) and one mixed culture were duplicate plated, grown overnight, and colonies counted. Results confirmed that the expected number of cells were present.

1 ml of the Listeria/Bacillus culture mix was treated with chitosan-biotin-streptavidin magnetic beads, prepared as in Example 1, as described in Example 2. As controls, 1 ml of Listeria only culture at 10² cfu/ml and 1 ml of Bacillus only culture at 10⁷ cfu/ml were also treated with chitosan-biotin-streptavidin magnetic beads. All samples were then washed and lysed as described in Example 1.

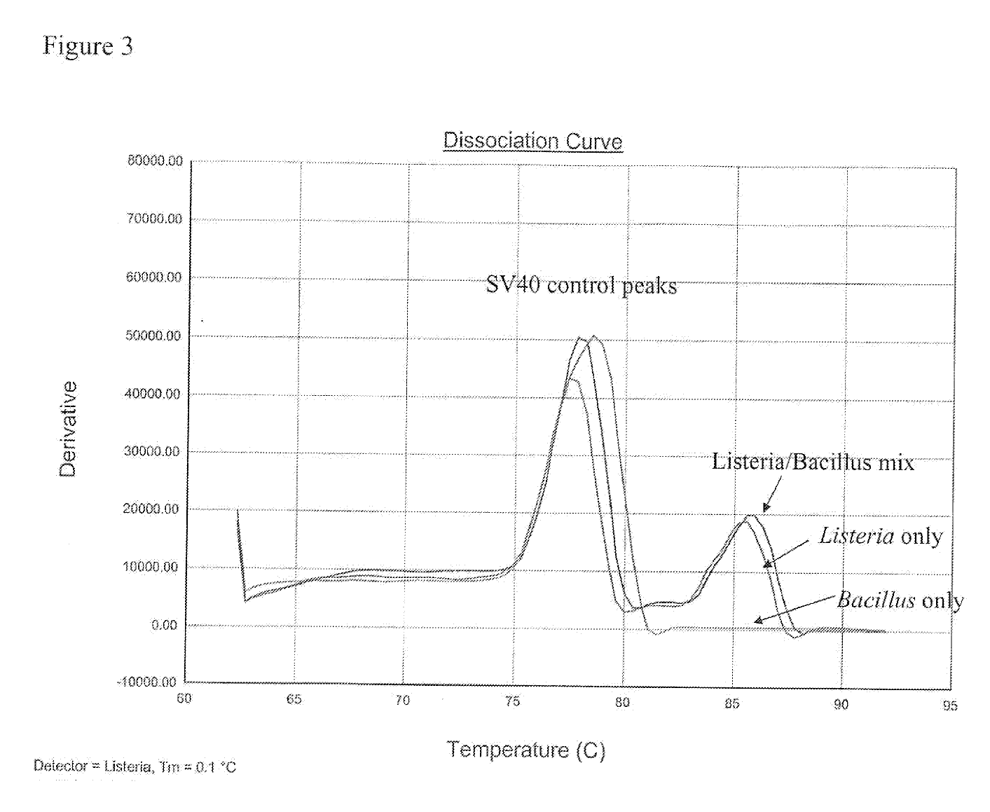

The presence of Listeria innocua was assayed using a Listeria genus BAX™ PCR detection kit from Qualicon as described in Example 1. FIG. 3 shows the results of the Listeria detection assay following chitosan capture as dissociation (melt) curves. Each of the three samples showed the control SV40 DNA peak. The Listeria DNA peak was present in the Listeria only control, as well as in the Listeria/Bacillus mix sample. This peak was not present in the Bacillus only control confirming the specificity of the assay. The results demonstrated that Listeria cells were captured by chitosan-biotin-streptavidin magnetic beads when present in a culture including multiple strains of bacteria. No interference in Listeria capture and detection was caused by the presence of a high background of non-target bacteria. Detection of the target bacteria was determined through the PCR reaction components, specifically the primers.

A sample of chitosan beads used for capture of the mixed Listeria/Bacillus culture was cultured on BHI plates. Both Listeria and Bacillus colonies were visually identified on these plates confirming that both types of bacteria were captured by the chitosan beads.

### Example 4

### Use of Different Molecular Weights of Chitosan Preparations in Biotin/Streptavidin Bead Linkage for Listeria Capture and Detection

Chitosan samples of two additional sizes: (S) and (L), were purchased from Primex (Siglufjordur, Iceland). The actual average molecular weights of the chitosan in each preparation was determined to be 34,400 for (S) and 180,000 for (L) using HPLC GPC-MALLS. Chitosan from each of these preparations as well as the 75,000 Dalton sample of Example 1 was derivatized with biotin as described in Example 1 except that the amount of Biotin-LC-NHS varied as given in Table 1.

TABLE 1
Calculation of NHS-LC-Biotin solution volume used in conjugation
reactions with different size of chitosan.

| Chitosan | | | | |
|---|---|---|---|---|
| Molec. | Moles of | [Biotin-LC- | | |
| Weight | chitosan | NHS] | Molar Ratio of | Vol of biotin |
| (Dalton) | (Moles) | (Mole/μl) | biotin/chitosan | (μl) |
| 33400 | 1.497 × 10⁻⁷ | 2.2 × 10⁻⁸ | 10 | 68.04 |
| 75000 | 6.667 × 10⁻⁸ | 2.2 × 10⁻⁸ | 10 | 30.30 |
| 180000 | 2.777 × 10⁻⁸ | 2.2 × 10⁻⁸ | 30 | 37.88 |

For each size of chitosan, approximately 120 μg of chitosan-biotin was attached to 1 ml of 1% Streptavidin/Dynal 2.8 μM magnetic beads, as described in Example 1, producing chitosan-biotin-streptavidin beads. Each of the three preparations of beads was used to capture Listeria innocua as described in Example 1, and the presence of Listeria detected by PCR also as described in Example 1.

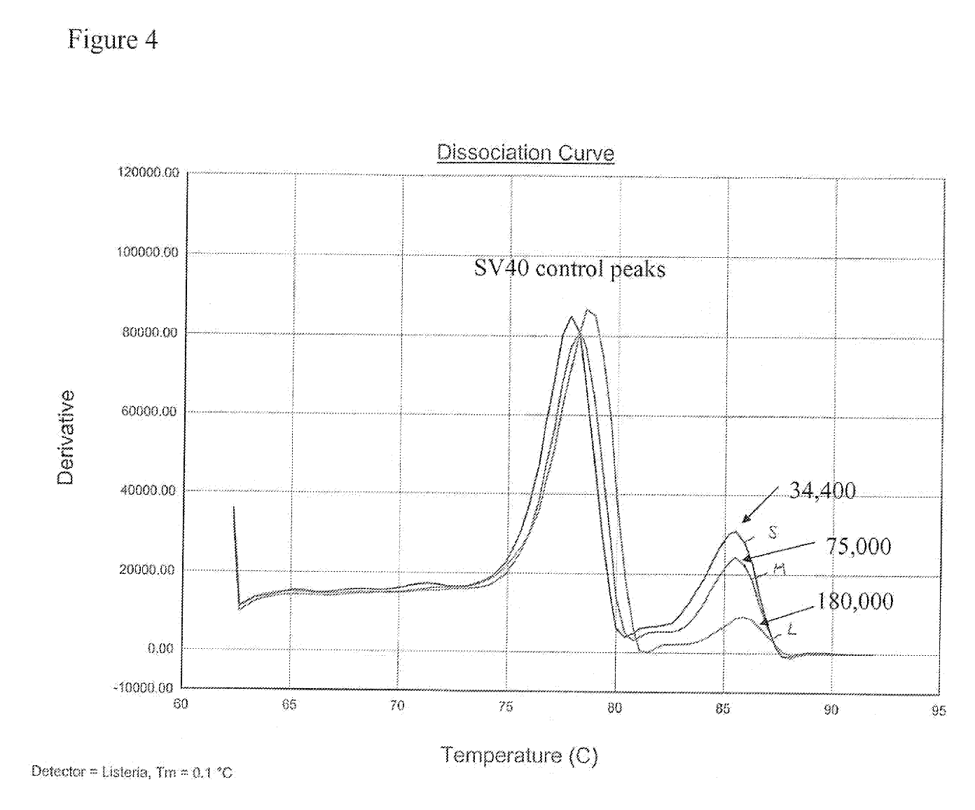

FIG. 4 shows the results of the Listeria detection assay following chitosan capture as dissociation (melt) curves. Each of the three samples showed the control SV40 DNA peak. The Listeria DNA peak was present in all samples which demonstrated that all three sizes of chitosan are able to capture Listeria cells for the detection assay. The 34,400 and 75,000 size chitosan showed similar efficiencies, while the 180,000 size chitosan was less effective. It was noted that there was more bead aggregation in the 180,000 chitosan sample, which may be related to a reduced effectiveness in bacterial cell capture.

### Example 5

### Direct Attachment of Chitosan to Magnetic Beads with p-toluenesulfonate and use for Capture of Listeria innocua for Detection Assay

Chitosan was directly attached to beads modified with p-toluenesulfonate (tosyl) as follows. Tosyl-activated 2.8 μM magnetic beads were purchased from Dynal Biotech ASA (Oslo, Norway). Aliquots of 0.5 ml of beads were washed 3 times with 0.5 ml of 0.1 M phosphate buffer, pH 7.4. The beads were gently mixed in the wash each time before separation on a magnetic rack and finally resuspended in 0.5 ml of phosphate buffer, pH 7.4. A 1% (10 mg/ml) solution of chitosan (75,000 Daltons) was prepared in 0.5% acetic acid as in Example 1. Based on the estimated amount of tosyl groups specified by the manufacturer, the amount of chitosan needed for a molar ratio of chitosan to tosyl groups of 10:1 was calculated. Chitosan solution and beads were mixed at this 10:1 ratio by adding 67 μl of the chitosan solution to 0.5 ml of beads. The sample mix was incubated at room temperature with rocking for 24 hr. Excess solution was removed from the samples placed on a magnetic rack, then the beads were washed twice with PBS, pH 5.75 including 1 mg/ml BSA, then stored at 4 ° C.

The chitosan-tosyl magnetic beads were used to capture cells from Listeria innocua cultures as described in Example 1, using cultures at 10⁴ cfu/ml and 10² cfu/ml. Lysis and detection were also as described in Example 1. Four separate and identical capture and detection assays were run. Chitosan-biotin-streptavidin beads prepared as in Example 1 were used for capture in separate samples at the same time for comparison, again with four replicates.

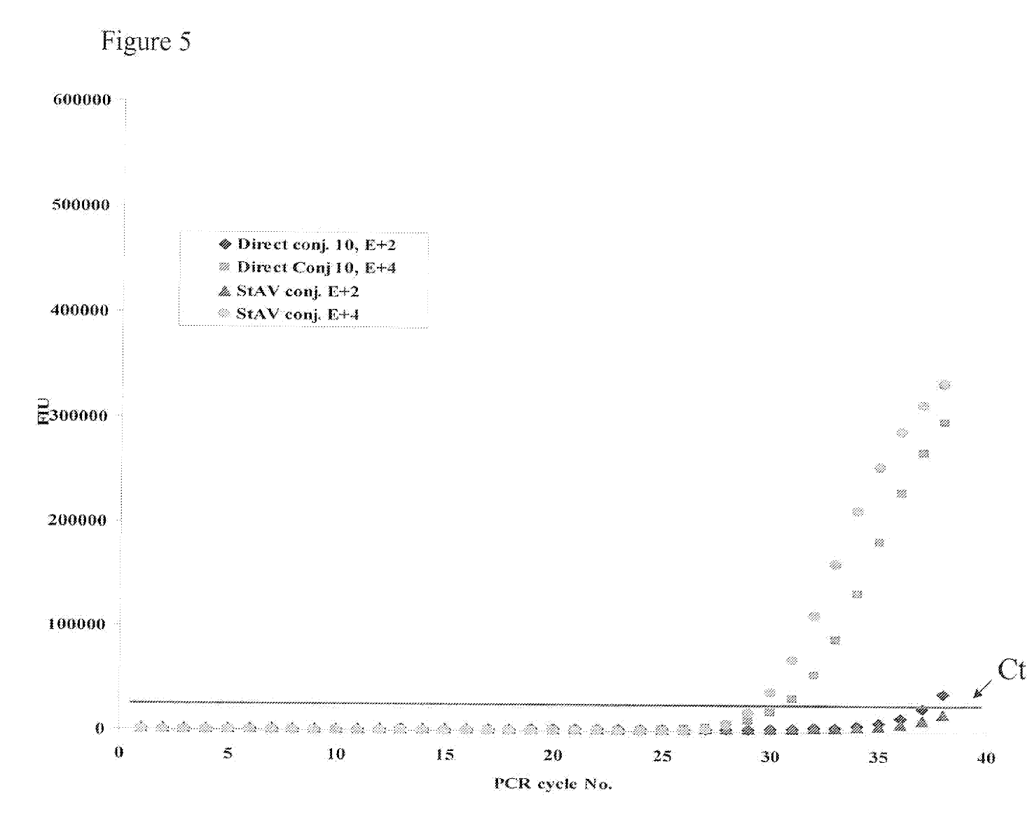

In all samples the Listeria genus DNA peak was seen in the dissociation curve at between 83.7 ° C. and 85.6 ° C., which indicated that Listeria cells were captured using the chitosan-tosyl beads as well as the chitosan-biotin-streptavidin beads. During thermocycling, Cycle threshold (Ct) values were determined for the formation of reaction product, and the average of the four identical assays for each bead type are shown in FIG. 5 and given in Table 2. Ct data from the ABI PRISM 7000 is inversely related to the abundance of the target template, with lower Ct values occurring when more template is present.

TABLE 2
Ct values for PCR of Listeria innocua captured with directly
and indirectly conjugated chitosan - Dynal magnetic beads.

| | Listeria innocua 10⁴ | Listeria innocua 10² |
|---|---|---|
| 2.8 μM magnetic | cfu/ml (N = 4); av. Ct | cfu/ml; av. Ct value of |
| beads (˜5 10⁷/assay) | value | four samples |
| Chitosan-biotin- | 26.88 | 33.48 |
| streptavidin- bead | | |
| Chitosan-tosyl-bead | 27.93 | 33.38 |

These results showed that the directly conjugated chitosan beads were effective in capturing bacterial cells for the PCR detection assay.

### Example 6

### Test of Bead Size in Direct Chitosan Attachment for Bacteria Capture and Detection

Chitosan of about 100,000 molecular weight (LV-25) was purchased from Primex (Siglufjordur, Iceland). The actual average size of the chitosan in this preparation was determined to be 99,530 Daltons using HPLC GPC-MALLS. Beads of three different sizes: 1μM, 2.8 μM and 4.5 μM, were purchased from Dynal Biotech ASA (Oslo, Norway). Based on the estimated amounts of tosyl groups specified by the manufacturer for each bead sample, the amounts of chitosan needed for a molar ratio of chitosan to tosyl groups of 12.5:1 were calculated. For the 1μM, 2.8 μM and 4.5 μM beads, 1% chitosan solution volumes of 0.29 ml, 0.25 ml, and 0.124 ml were used, respectively. Chitosan was attached to the beads as described in Example 5 except that 1 ml of each bead sample was used. The final bead concentration in each size of bead sample with chitosan attached was determined using the Particle Measuring System Liquilaz/APSS LS200 (Particle Measure system, Inc; Boulder, Colo., USA ) which counts each particle as it passes through a laser beam.

Listeria innocua culture samples were captured using 30 μl of each of undiluted, one to 10 diluted, and one to 100 diluted beads with chitosan attached and assayed as described in Example 1. Results of PCR assays are given as Ct values in Table 3.

TABLE 3
Study of different bead size and bead dilution factors in chitosan
captured Listeria innocua determined by Ct value of PCR assay.

| | | PCR Ct | | |
|---|---|---|---|---|
| Bead size | Beads/ml in | Undil. | PCR Ct | PCR Ct |
| μM | undiluted | beads | 1/10 dil. | 1/100 dil. |
| 1 | ˜3.3 × 10⁹ | 27.61 | 28.97 | 33.04 |
| 2.8 | ˜2 × 10⁹ | 28.47 | 31.44 | 35.42 |
| 4.5 | ˜8.7 × 10⁸ | 27.0 | 29.61 | 35.13 |

The results showed no significant difference in Ct value for bead size. Thus bead size is not critical for bacteria capture for detection using attached chitosan. Samples where capture was carried out with diluted beads, for all three sizes, did have higher Ct values indicating less efficient detection in the PCR assay. The 1:10 dilution showed a small increase in Ct value, while the 1:100 dilution showed a further increase. This result indicates that reducing the bead number reduced the amount of bacteria captured, which is consistent with a lower amount of chitosan available to bind the bacteria.

### Example 7

Detection of Multiple Targets in Chitosan Captured Samples

Chitosan capture was shown to be efficient for detection of two different target nucleic acid species in Listeria moncytogenes. Listeria moncytogenes was purchased from the ATCC (#15313) and grown as described in Example 1. Samples were prepared using cultures at 10⁴ cfu/ml and captured on chitosan-biotin-streptavidin beads as described in Example 1. Captured samples were lysed and assayed using the Listeria genus PCR kit as described in Example 1. Captured samples were also assayed using the BAX™ Listeria monocytogenes PCR kit (Qualicon, Wilmington, Del.), with the same conditions. An additional control sample contained buffer only.

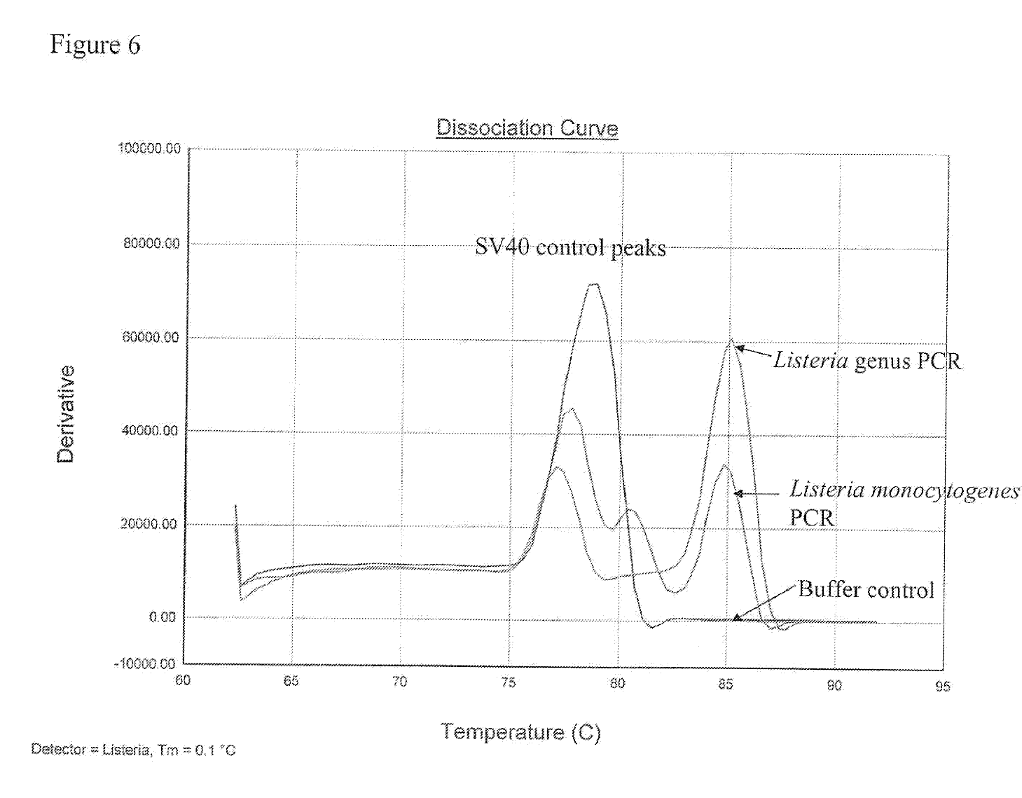

FIG. 6 shows the results of the detection assay following chitosan capture as dissociation (melt) curves. Each of the three samples showed the control SV40 DNA peak. Both the Listeria genus PCR and the Listeria monocytogenes PCR assays gave a strong peak at about 85° C., with no peak at this temperature in the buffer control. These results indicated that primers in both the Listeria genus and the Listeria monocytogenes assay kits were able to amplify a product from the chitosan captured sample. Since the primers of the two kits target amplification of different DNA sequences, this example demonstrates that multiple DNA species may be assayed from the same chitosan captured sample.

What is claimed is:

1. A method for detection of a microorganism in a sample comprising:
a) providing a support coated with chitosan;
b) providing a sample having at least one microorganism strain;
c) contacting the coated support of (a) with the sample of (b), whereby the at least one microorganism strain is bound to the support; and
d) detecting the presence of the at least one bound microorganism strain.

2. A method for detection of a microorganism in a sample comprising:
a) providing a support coated with chitosan;
b) providing a sample containing a sample matrix and suspected of containing at least one target microorganism strain;
c) contacting the coated support of (a) with the sample of (b) such that at least one target microorganism strain is bound to the support;
d) washing the sample matrix from the bound target microorganism strain;
e) lysing the bound target microorganism strain to release nucleic acids; and
f) detecting the presence of at least one of said nucleic acids by at least one primer directed amplification method.

3. A method according to claim 1, wherein the support is selected from the group consisting of at least one of a bead, a film, a sheet, a particle, a filter, a membrane, a plate, a strip, a tube, a well, a gel, fibers, capillaries and combinations of all of these.

4. A method according to claim 3, wherein the bead is a magnetic bead.

5. A method according to claim 4, wherein the magnetic bead is about 1 to about 5 microns in diameter.

6. A method according to claim 1, wherein the support comprises materials selected from the group consisting of glass, silica, latex, ceramics, metals, polyolefins, polyolefin copolymers, polyolefin ionomers, polyolefin blends, poly(4-methylbutene), polystyrene, polymethacrylate, polyethylene, polypropylene, polyamide (nylon), poly(vinyl butyrate), poly(ethyleneterephthalate) (PET), polyvinylchloride (PVP), polycarbonate, polyesters, and cellulose.

7. A method according to claim 1, wherein the chitosan is of molecular weight in the range of about 2,000 to about 500,000 Daltons.

8. A method according to claim 1, wherein the chitosan is directly linked to the support.

9. A method according to claim 1, wherein the chitosan is linked to the support via a linker or attachment molecule.

10. A method according to claim 9, wherein the attachment molecule is one member of a binding pair independently selected from the group consisting of antigen/antibody, hapten/anti-hapten, IgG/protein A, IgG/protein G, biotin/avidin, biotin/streptavidin; glutathione-S-transferase/glutathione and folic acid/folate binding protein.

11. A method according to claim 9, wherein the linker is p-toluenesulfonate and the attachment molecule is streptavidin.

12. A method according to claim 1, wherein the sample contains a plurality of microorganism strains.

13. A method according to claim 12, wherein any one strain from the plurality of microorganism strains is detected independently of any other strain.

14. A method according to claim 1, wherein the microorganism is selected from the group consisting of bacteria, fungi, yeasts and enveloped viruses.

15. A method according to claim 14, wherein the microorganism is a pathogen.

16. A method according to claim 14, wherein the pathogen is selected from the group of genera consisting of Listeria, Escherichia, Salmonella, Shigella, Campylobacter, Clostridium, Helicobacter, Mycobacterium, Staphylococcus, Enterococcus, Bacillus, Neisseria, Shigella, Streptococcus, Vibrio, Yersinia, Bordetella, Borrelia, and Pseudomonas.

17. A method according to claim 1, wherein the sample is selected from the group consisting of medical, environmental, food, feed, clinical and laboratory samples.

18. A method according to claim 17, wherein the environmental sample is from an environment selected from the group consisting of soil, water, medical environment, veterinary environment, food environment, food preparation environment, industrial environment, terrorism suspect environment, and laboratory environment.

19. A method according to claim 1, wherein detection of the presence of the microorganism is by genetic detection.

20. A method according to claim 19, wherein the genetic detection comprises nucleic acid hybridization.

21. A method according to claim 19, wherein the genetic detection comprises a primer directed amplification method.

22. A method according to claim 21, wherein the primer directed amplification method comprises a method selected from the group consisting of polymerase chain reaction, reverse transcriptase polymerase chain reaction, ligase chain reaction, strand displacement amplification and combinations of these.

23. A method according to claim 2, wherein the primer directed amplification method comprises a method selected from the group consisting of polymerase chain reaction, reverse transcriptase polymerase chain reaction, ligase chain reaction, strand displacement amplification and combinations of these.

24. A method for detection of a microorganism in a sample comprising:
a) providing a support coated with chitosan;
b) providing a sample containing a sample matrix and suspected of containing at least one target microorganism strain;
c) contacting the coated support of (a) with the sample of (b), such that the at least one microorganism strain is bound to the support;
d) washing the sample matrix from the bound target microorganism strain;
e) detecting the presence of said microorganism strain by detection methods selected from the group consisting of genetic detection methods and immunologic detection methods.

25. A microorganism capture device comprising a support comprising chitosan.

26. A device according to claim 25 wherein the support is selected from the group consisting of at least one of a bead, a film, a sheet, a particle, a filter, a membrane, a plate, a strip, a tube, a well, a gel, fibers, capillaries and combinations of all of these.

27. A device according to claim 26 wherein the bead is a magnetic bead.

28. A device according to claim 25 wherein the support is comprised of materials selected from the group consisting of glass, silica, latex, ceramics, metals, polyolefins, polyolefin copolymers, polyolefin ionomers, polyolefin blends, poly(4-methylbutene), polystyrene, polymethacrylate, polyethylene, polypropylene, polyamide (nylon), poly(vinyl butyrate), poly(ethyleneterephthalate) (PET), polyvinylchloride (PVP), polycarbonate, polyesters, and cellulose.

29. A diagnostic kit comprising the capture device of claim 25.
